SEQUENCE LISTING (6 sequences)

SEQ ID NO: 1 - DNA, 6198 bp, Artificial Sequence
  baculovirus L1X plasmid encoding for mutant HPV16/31L1 (pFastbac)
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatccatgagtctctggctcccctcggaggcaaccgtatacctccctcccgtcccagtgtctaaagtggtgtctaccgacgagtacgtcgcaagaactaacatctactaccatgccggcacttcacgtcttttggccgtgggacatccttactttccgattaagaagccaaacaacaataagattcttgtcccaaaagtttcgggtttgcaataccgcgttttccgcatccacctccgcgatccgaataagttcggcttcccagacacgtccttttacaatccggacactcaacgtttggtgtgggcctgtgtgggagtggaggtgggtcgtggacaaccgttgggcgttggaatttccggtcatcccctccttaacaagttggatgacaccgaaaatgcatcagcatacgctgcaaacgccggagtagataaccgcgagtgtatctctatggactataagcagacgcagctctgcctgattggttgtaagcctccaattggtgagcactggggcaaaggaagcccctgcaataacgtagccgtgaaccccggtgactgccctcctctggagctgataaacacggtcatccaagacggagatatggtcgataccggtttcggagctatggatttcactactctccaggctaacaagtccgaagtcccattggatatctgtacctcgatatgcaaataccccgattacatcaagatggttagcgaaccctacggcgactcactgttcttctatttgaggagagaacaaatgttcgtccgtcacctcttcaacagagctggtgcggtaggcgagaacgtccctacagacctctacatcaagggttctggtagcacagcgactctggcgaattcaaactatttccccactcccagtggaagcatggtgacctcagacgcccagatcttcaataagccctattggcttcagcgtgctcaaggccacaacaacggtatctgctggggcaatcaactgttcgtcacagttgtcgataccacgagatctaccaatatgtcgttgtgcgctgcgatttctacgtccgaacctacttacaagaacaccaacttcaaggagtacttgaggcatggtgaagaatacgatctgcaattcatcttccagctgtgcaagataacgctcaccgctgacgtaatgagctacatccactctatgaacagcactatcttggaggactggaactttggcctccagccgcctccaggcggaaccctggaggacacatatcgctttgttacctcccaggcgattgcttgccagaagcacacacctcctgctcccaaggaggaccctctcaagaaatacacattttgggaggtcaacttgaaagaaaagtttagtgccgatctggaccagtttcccttgggtaggaaattcctgctgcaggccggtctgaaggctaagccgaaattcacacttggcaagcgtaaagccactccaaccactagttccacctcaacaacagctaaacgtaagaagaggaaactttagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 2 - DNA, 6102 bp, Artificial Sequence
  baculovirus L2 plasmid encoding for HPV16L2 (pfastbac)
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatccatgcgccacaagaggtctgctaaacgtaccaaaagagcttctgcaactcagctctacaagacatgcaagcaggcaggcacgtgtcctcccgacatcattcccaaggtcgagggaaagaccattgctgatcaaatccttcagtacggatcgatgggcgtgttcttcggaggtctgggcattggtaccggttccggcacgggcggacgcaccggatacatccctcttggtactcgtcctcccacggccactgacacactggctcctgtccgtcctccgctcacagtggaccccgttggccctagtgacccctccatcgtcagccttgtggaagaaaccagctttatcgatgcgggagctcctactagcgttccatctatccctccggacgtgagcggtttctctatcactacttcaaccgatacaactcccgcgatcctcgatatcaacaacacggtcacgactgtcacaacccataacaatcctacttttaccgatccatcggtactgcaaccgcccacccctgctgaaaccggcggtcacttcacactgtcgtcatcaactatcagcactcataactacgaggagatcccgatggatacgttcatcgtgtcgaccaatcccaatactgttacctcctcaacccctatcccgggaagtaggcctgtagccaggttgggcctttacagtagaaccactcagcaggtcaaagtagttgaccctgcctttgttacaacacccaccaagttgattacctacgacaacccagcatacgagggcattgatgtcgataacacactctacttctcctctaacgacaatagcatcaatatcgctccagaccccgactttctggacatcgtcgccctgcaccgtcccgcactgacctcacgtaggaccggtatcagatattctcgcattggaaacaaacaaaccttgcgtactaggtctggcaagagcataggagcgaaggtacactattactatgatctctctacaatcgatccagctgaggagatcgaactccagacgattacgccgtccacatatactacgacttcccacgccgcatcacctacatccatcaacaacggcttgtacgacatctacgccgacgacttcatcactgatacttcgaccaccccagtgccatccgtgccatccacttctttgagtggttacatacccgccaataccactattcccttcggtggtgcctacaacattccactggtgtccggacccgacattcctatcaacatcacggaccaagccccttcacttattccaatagtacccggtagtccgcagtataccatcatagcggatgcgggcgacttctatctccatccaagttactacatgttgcgcaagcgccgcaagagactgccatacttcttctccgacgtgagcctggctgcttgatagaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 3 - DNA, 49 bp, Artificial Sequence
  shE7-1 Forward
caccaggaggatgaaatagatggttcgaaaaccatctatttcatcctcc

SEQ ID NO: 4 - DNA, 49 bp, Artificial Sequence
  shE7-1 Reverse
aaaaggaggatgaaatagatggttttcgaaccatctatttcatcctcct

SEQ ID NO: 5 - DNA, 10827 bp, Artificial Sequence
  Plasmid p16L1*L2 encoding 16/31 L1 (L1*) and L2 human codon optimized
ctagagccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccaccgacctgtacatcaagggcagcggcagcaccgccaccctggccaacagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagcttatcgataccgtcgacctcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgaaagctttttgaattctttggatccactagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggctagagtacttaatacgactcactatagg

SEQ ID NO: 6 - DNA, 10827 bp, Artificial Sequence
  p16sheLL plasmid sequence
ctagagccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagcttatcgataccgtcgacctcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgaaagctttttgaattctttggatccactagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggctagagtacttaatacgactcactatagg

(19) United States
(12) Patent Application Publication
(10) Pub. No.: US 2013/0012426 A1
(43) Pub. Date: Jan. 10, 2013

(54) Virion Derived Protein Nanoparticles For Delivering Diagnostic Or Therapeutic Agents For the Treatment of Psoriasis

(75) Inventor: Elisabet de los Pinos, Brookline, MA (US)

(73) Assignee: AURA Biosciences, Inc., Cambridge, MA (US)

(21) Appl. No.: 13/273,206

(22) Filed: Oct. 13, 2011

Related U.S. Application Data
(60) Provisional application No. 61/506,140, filed on Jul. 10, 2011.

(51) Int. Cl. A61K 38/16; A61P 35/00

(52) U.S. Cl. 514/1.2

(57) ABSTRACT

This invention relates to a transdermal delivery system for treating skin related diseases employing protein nanoparticles to deliver drugs to the keratinocytes and basal membrane cells for the treatment of Psoriasis. The current invention presents an effective method for delivering small molecule nucleic acids to the epidermal cells.

### RELATED APPLICATIONS

This application claims the benefit of priority to U.S. Provisional Application No. 61/506,140 filed Jul. 10, 2011. The disclosures of the above applications are incorporated herein by reference.

### REFERENCE TO SEQUENCE LISTING

The Sequence Listing provides exemplary polynucleotide sequences of the invention. The traits associated with the used of the sequences are included in the Examples.

The Sequence Listing submitted as an initial paper is named AURA_{—}16_ST25.txt, is 45 kilobytes in size, and the Sequence Listing was created on 29 Nov. 2011. The copies of the Sequence Listing submitted via EFS-Web as the computer readable for are hereby incorporated by reference in their entirety.

### FIELD OF INVENTION

The invention relates to methods for loading protein nanoparticles with therapeutic, diagnostic or other agents, wherein the protein nanoparticles are based on viral proteins. More particularly, the present invention relates to a method for using protein nanoparticles to deliver drugs to the keratinocytes and basal membrane cells for the treatment of Psoriasis.

### BACKGROUND OF THE INVENTION

Ribonucleic acid (RNA) is one of the three major macromolecules (along with DNA and proteins) that are essential for life. Messenger RNA (or “mRNA”) is a type of RNA molecule that carries genetic information from DNA to produce proteins. mRNA is the intermediary for the production of proteins within the body, and each specific mRNA directs the production of a specific protein.

Another type of RNA molecule called small interfering RNA (“siRNA”) does not lead to the production of proteins, but instead interferes with the production of proteins. siRNA does so by binding itself to a particular mRNA molecule, which leads to the destruction of the mRNA. Through this targeted destruction of particular mRNA molecules, the siRNA interferes with the production of the protein that would otherwise have been produced by the mRNA molecule.

The process of siRNA targeting mRNA molecules occurs naturally and plays an important role in regulating the production of proteins in the body, and in protecting against infectious diseases. For example, some viruses use RNA as their genetic material. siRNA molecules can bind themselves to RNA viruses and target them for destruction, and in so doing disrupt the course of viral infections.

In the RNA interference (“RNAi”) field, scientists have researched ways to use siRNA to combat diseases, such as by attempting to create specially-tailored siRNA drugs to “turn off” the production of proteins associated with diseases or viruses.

This requires not only identifying, designing, and modifying siRNA sequences for use in the drug, but also developing a delivery system to deliver the siRNA molecule safely and efficiently to its intended destination in the body. Although scientists have had success developing siRNA molecules to use in these types of drugs, it has been far more difficult to figure out how to deliver siRNA molecules to their target sites efficiently and safely through the bloodstream or skin.

Delivering siRNA poses several complex challenges. First, the siRNA has to survive transport to disease sites without degradation. Second, the siRNA must be sufficiently shielded from components of the immune system during transport to avoid unwanted immune effects. Third, the siRNA must actually reach its intended target within the body. Fourth, once the siRNA reaches its intended target, it must be efficiently released into the interior of the cells of the target tissue. Adding to the challenge, all of the above must occur at an appropriate rate and level to achieve the best therapeutic outcome.

With respect to delivering siRNA through the epidermis, a variety of transdermal delivery methods have been explored, but to date, intradermal injections continue to be the most effective. This is despite the fact that clinical trials with intradermal injections have been discontinued due to the pain of this treatment option. (Leachman 2009) Further, although effective knockdown of targeted gene expression has been determined, the effects have been localized to the injection site. (Leachman 2009). Finally, it is known that delivering siRNA through the stratum corneum is necessary but it is also known that this path is not sufficient for delivery to epidermal cells and that additional steps must be taken to facilitate nucleic acid uptake by keratinocytes (and endosomal release) to allow access to the RNA-induced silencing complex.

Psoriasis is a chronic inflammatory skin disorder affecting 1-3% of the world population. The disease is characterized by demarcated erythematous scaly plaques in which keratinocytes exhibit hyperproliferation and abnormal differentiation leading to epidermal hyperplasia.

The cause of psoriasis is not fully understood. There are two main hypotheses about the process that occurs in the development of the disease. The first considers psoriasis as primarily a disorder of excessive growth and reproduction of skin cells. The problem is simply seen as a fault of the epidermis and its keratinocytes. The second hypothesis sees the disease as being an immune-mediated disorder in which the excessive reproduction of skin cells is secondary to factors produced by the immune system. T cells (which normally help protect the body against infection) become active, migrate to the dermis and trigger the release of cytokines (tumor necrosis factor-alpha TNFα, in particular) which cause inflammation and the rapid production of skin cells. It is not known what initiates the activation of the T cells.

There are a number of different treatment options for psoriasis. Typically topical agents are used for mild disease, phototherapy for moderate disease, and systemic agents for severe disease.

Systemic Agents

Psoriasis that is resistant to topical treatment and phototherapy is treated by medications taken internally by pill or injection (systemic). Patients undergoing systemic treatment are required to have regular blood and liver function tests because of the toxicity of the medication. Most people experience a recurrence of psoriasis after systemic treatment is discontinued.

The three main traditional systemic treatments are methotrexate, cyclosporine and retinoids. Methotrexate and cyclosporine are immunosuppressant drugs; retinoids are synthetic forms of vitamin A. Patients taking methotrexate are prone to ulcerations. Post-surgical eventration may be associated to methotrexate exposure.

Biologics are manufactured proteins that interrupt the immune process involved in psoriasis. Unlike generalised immunosuppressant therapies such as methotrexate, biologics focus on specific aspects of the immune function leading to psoriasis. These drugs (interleukin antagonists) are relatively new, and their long-term impact on immune function is unknown, but they have proven effective in treating psoriasis and psoriatic arthritis. Biologics are usually given by self-injection or in a doctor's office.

Two biologics known to target T cells are efalizumab and alefacept. Efalizumab is a monoclonal antibody which blocks the molecules that dendritic cells use to communicate with T cells. It also blocks the adhesion molecules on the endothelial cells that line blood vessels, which attract T cells. However, it suppresses the immune system's ability to control normally harmless viruses, which can lead to fatal brain infections. Efalizumab was voluntarily withdrawn from the US market in April, 2009 by the manufacturer. Alefacept also blocks the molecules that dendritic cells use to communicate with T cells and even causes natural killer cells to kill T cells as a way of controlling inflammation (Nestle F O, Kaplan D H, Barker J (July 2009). “Psoriasis”. N. Engl. J. Med. 361 (5): 496-509).

Several monoclonal antibodies (MAbs) target cytokines, the molecules that cells use to send inflammatory signals to each other. TNF-α is one of the main executor inflammatory cytokines. Four MAbs adalimumab, golimumab and certolizumab pegol) and one recombinant TNF-α decoy receptor, etanercept, have been developed against TNF-α to inhibit TNF-α signaling. Additional monoclonal antibodies have been developed against pro-inflammatory cytokines IL-12/IL-23 and Interleukin-17 (Hueber W, Patel D D, Dryja I, et al. (October 2010).

In 2008, the FDA approved three new treatment options available to psoriasis patients: 1) Taclonex Scalp, a new topical ointment for treating scalp psoriasis; 2) the Xtrac Velocity excimer laser system, which emits a high-intensity beam of ultraviolet light for treating moderate to severe psoriasis; and 3) the biologic drug adalimumab (brand name Humira) was also approved to treat moderate to severe psoriasis. Adalimumab had already been approved to treat psoriatic arthritis. The most recent biologic drug that has been approved to treat moderate to severe psoriasis, as of 2010, is ustekinumab (brand name Stelara).

Despite the growing number of treatment options, extensive use of therapeutic monoclonal antibodies in clinic has shown that toxic effects may occur in patients. These undesirable effects can be classified in four categories: cytokine release syndrome, auto-immune diseases, organ toxicity and opportunistic infections. Immunogenicity, which is highly variable depending on the degree of humanization, could also potentially lead to adverse effects due to immune-complexes formation. A recent accident observed with the anti-CD28 TGN1412 has led to the conclusion that the relative confidence in the safety of monoclonal antibodies should be revised.

Accordingly, there is an unmet need for delivery strategies that increase bioavailability, selectivity and targeting of drugs to treat Psoriasis.

### SUMMARY OF INVENTION

The object of the present invention is to overcome the shortcomings disclosed in the prior art. More specifically, the present invention provides particles and methods for using pseudo-viruses, including those derived from the herpes and human papilloma viruses, to deliver siRNA to keratinocytes and basal membrane cells for the treatment of Psoriasis.

The accompanying drawings, which are incorporated in and constitute part of the specification, illustrate various embodiments of the invention and together with the description, serve to explain the principles of the invention.

### BRIEF DESCRIPTION OF THE SEQUENCE LISTINGS AND DRAWINGS

FIG. 1 shows a flow chart diagram of a preferred embodiment of the present invention.

FIG. 2 depicts shuttle vector information.

FIG. 3 depicts L1 capsid protein in various fractions from insect cell culture (T=total cell lysate, C=cytoplasmid fraction, TN=total nuclear fraction, SN=soluble nuclear fraction). Harvest times after baculovirus infection indicated.

FIG. 4 shows results from in vitro reassembly of capsid protein produced in insect cell culture. DLS demonstrates presence of capsid protein in form of monomers and oligomers after harvest from nuclear fraction (left) and appearance of well formed loaded VLPs after the reassembly procedure (right).

FIG. 5 is a graph showing the amount of luminescence/luciferase signal measured 48 hrs after treatment of HeLa cells with loaded VLP, where luminescence is reported on a scale of 0 to 30,000 units along the y-axis.

FIG. 6 is a graph the same data in FIG. 1I A, showing the amount of luminescence/luciferase signal measured 48 hrs after treatment of HeLa cells with loaded VLP, where luminescence is reported on a scale of 0 to 20 units along the y-axis.

(SEQ ID NO: 1) shows DNA sequence for baculovirus L1X plasmid encoding HPV16/31L1 (pFastBac™).

(SEQ ID NO: 2) shows DNA sequence for baculovirus L2 plasmid encoding HPV16L2 (pFastBac™).

(SEQ ID NO: 3) shows forward primer DNA sequence used for generation of shE7-1 RNA construct.

(SEQ ID NO: 4) shows reverse primer DNA sequence used for generation of shE7-1 RNA construct.

(SEQ ID NO: 5) shows plasmid p16L1*L2 DNA sequence encoding 16/31 L1 (L1*) and L2 human codon-optimized.

(SEQ ID NO: 6) shows p16sheLL plasmid DNA sequence.

### DETAILED DESCRIPTION OF THE INVENTION

The present invention provides both the barrier disruption and the intracellular delivery that has been long needed for the delivery of nucleic acids to the skin. Herpes and human papilloma viruses as delivery vehicles have the inherent characteristics to overcome the stratum cornea barriers and efficiently provide intracellular delivery of the nucleic acid payload.

In accordance with a first preferred embodiment of the present invention, a method for topically treating Psoriasis using a combination of betapapillomavirus viral shells (L1/L2) to deliver a siRNA against cytokines is provided.

A first step in this preferred embodiment includes constructing a recombinant DNA molecule that contains a sequence encoding a papillomavirus L1 protein or a papillomavirus L2 protein or a combination of L1 and L2 proteins and then transfecting a host cell with the recombinant DNA molecule. Preferably, the virus like particles may express papillomavirus L1 protein or L2 protein or a combination of L1 and L2 proteins in the host cell. Next, the betapapillomavirus virus-like particles obtained from the transfected host cell may be purified which will cause the disassembling of the L1 and L2 capsid proteins of the virus-like particles into smaller units. Preferably, it is these smaller disassembled L1 and L2 capsid proteins which may he loaded with a siRNA against cytokines. Next the loaded proteins may be reassembled to form loaded virus-like particles comprising HPV protein with the siRNA against cytokines and administered to the skin of an animal or a human subject.

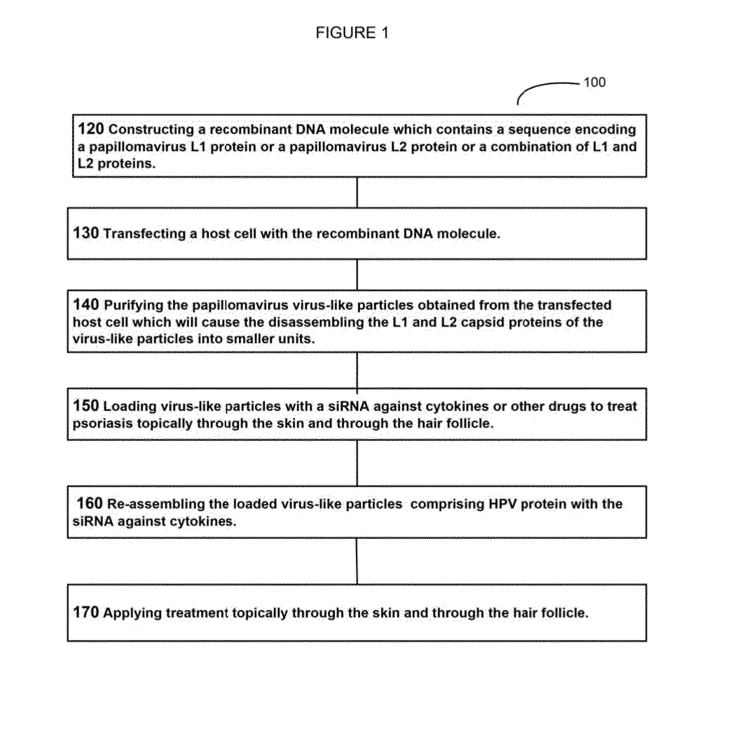

With reference now to FIG. 1, a method in accordance with an embodiment of the present invention will now be discussed. As shown in FIG. 1, the present invention provides a method for treating Psoriasis 100, which includes a first step in which a recombinant DNA molecule is contructed which contains a sequence for encoding a papillomavirus L1 protein or a papillomavirus L2 protein, or a combination of papillomavirus L1 and L2 proteins 120. Thereafter, a host cell will be transfected with the recombinant DNA molecule 130. After which, the transfected host cell will be treated to purify the papillomavirus virus-like particles causing the L1 and L2 capsid proteins to disassemble into smaller units 140. At which time, an appropriate therapeutic agent or drug for treating Psoriasis will be introduced into the proximity of the virus-like particle where the agent or drug for treatment will be loaded into the virus-like particles 150. Thereafter, the loaded virus-like particles enclosing siRNA against cytokines may be reassembled 160. Finally, the treatment may preferably be topically applied through the skin 170 for the treatment of psoriasis.

Preferably, according to one aspect of the present invention, the RNA which inhibits the expression of cytokines is an siRNA which inhibits the expression of TNF-α for the treatment of psoriasis.

Assembly of Particles

To assemble the biological, pharmaceutical or diagnostic components to a described biological cargo-laden nanoparticles used as a carrier, the components can be associated with the nanoparticles through a linkage. By “used as a carrier associated with,” it is meant that the component is carried by the nanoparticles. The component can be dissolved and incorporated in the nanoparticles non-covalently. Preferred and illustrative methods for creating, loading and assembling particles for use with the present are taught in following applications which are hereby incorporated by reference in their entirety: WO2010120266 entitled “HVP PARTICLES AND USES THEREOF;” WO2011039646, Nov. 24, 2010 entitled “TARGETING OF PAPILLOMA VIRUS GENE DELIVERY PARTICLES;” U.S. Provisional Application No. 61/417,031 entitled “METHOD FOR LOADING HPV PARTICLES;” and U.S. Provisional Application No. 61/491,774 entitled “PAPILLOMA-DERIVED PROTEIN NANOSPHERES FOR DELIVERING DIAGNOSTIC OR THERAPEUTIC AGENTS.”

In some embodiments, aspects of the invention relate to methods and compositions for producing protein nanoparticles that contain therapeutic and/or diagnostic agents for delivery to a subject. Methods and compositions have been developed for effectively encapsulating therapeutic and/or diagnostic agents within papilloma virus proteins (e.g., HPV proteins) that can be used for delivery to a subject (e.g., a human subject). Alternatively, other virus proteins may be used as delivery agents within the scope of the present invention. For instance, herpes viral vectors may be used as delivery agents.

In some embodiments, it has been discovered that it is useful to isolate L1 and L2 capsid proteins directly from host cells as opposed to disassembling VLPs that were isolated from host cells. L1 and L2 capsid proteins that are isolated directly from cells can be used in in vitro assembly reactions to encapsulate a therapeutic or diagnostic agent. This avoids the additional steps of isolating and disassembling VLPs. This also results in a cleaner preparation of L1 and L2 proteins, because there is a lower risk of contamination with host cell material (e.g., nucleic acid, antigens or other material) that can be contained in VLPs that are isolated from cells.

In some embodiments, it has been discovered that expressing L1 and/or L2 proteins intracellularly in the presence of a therapeutic or diagnostic agent can be useful in the production of a loaded VLP intracellularly that encapsulates the agent.

In some embodiments, it is useful to independently produce L1 and L2 capsid proteins. In some embodiments, they can be produced from two independent nucleic acids (e.g., different vectors). In some embodiments, they can be produced in the same cell (e.g., using two different vectors within the same cell). In some embodiments, they can be produced in different cells (e.g., different host cells of the same type or different types of host cell). This approach allows the ratio of L1 and L2 proteins to be varied for either in vitro or intracellular assembly. This allows VLPs to be assembled (e.g., in vitro or intracellularly) with higher or lower L1 to L2 ratios than in a wild type VLP. This may have benefits in the use of HPV nanoparticles as delivery vehicles for therapeutic agents. A higher ratio of L2 in the assembled structure may allow the resultant VLP to have a higher nucleic acid binding affinity and a better efficiency in delivering these intracellularly.

Capsid Proteins:

In some embodiments, L1 and L2 proteins are expressed in a host cell system (e.g., both in the same host cell or independently in different host cells). L1 and/or L2 are isolated from nuclei of the host cells. In some embodiments, certain L1 and/or L2 structures that are formed during cellular growth (e.g., during the fermentation process) are disrupted. Any suitable method may be used. In some embodiments, sonication may be used (e.g., nuclei may be isolated and then sonicated). Capsid proteins then may be purified using any suitable process. For example, in some embodiments, capsid proteins may be purified using chromatography.

Isolated capsid proteins can then he used as described herein in a cell free system to assemble together with different payloads to create superstructures that contain a drug or diagnostic agent in its interior.

It should be appreciated that directly isolating capsid proteins (as opposed to isolating and disassembling VLPS) provides several benefits. In some embodiments, there is a reduced risk of encapsulating and transferring genetic information (DNA, RNA) from the host cell to the treated subject. In certain embodiments, de-novo assembly of VLPs during the assembly procedure ensures formation of a larger percentage of loaded VLPs as opposed to using already-formed VLPs for loading where a certain fraction can remain unloaded.

Cellular Production:

In some embodiments, one or more therapeutic or diagnostic agents may be loaded intracellularly by expressing L1 and/or L2 in the presence of intracellular levels of one or more agents of interest.

In some embodiments, this method is used for encapsulating a silencing plasmid which will encode for expression of short hairpin RNA (shRNA). In some embodiments, this plasmid will have a size of 2 kB-6 kB. However, any suitable size may be used. In some embodiments, a plasmid is designed to be functional within the cells of the patient or subject to be treated (to which the loaded VLP is administered). Accordingly, the plasmid will be active within the target cells resulting in knockdown of the targeted gene(s).

In some embodiments, this method may be used to encapsulate short interfering RNA (siRNA) or antisense nucleic acids (DNA or RNA) transfected into the host cells (e.g., 293 cells or other mammalian or insect host cells) during the production of the VLPs.

Accordingly, loaded VLPs may be produced intracellularly to provide gene silencing functions when delivered to a subject.

It should be appreciated that there are several benefits to this method. In some embodiments, encapsulation of RNA interference (RNAi) constructs into VLPs allows for very efficient transfer of RNAi or Antisense nucleic acid into target cells.

Independent Expression Vectors:

In some embodiments, L1 and L2 proteins are expressed in a host cell system (e.g. mammalian cells or insect cells) from independent expression nucleic acids (e.g., vectors, for example, plasmids) as opposed to both being expressed from the same nucleic acid.

It should be appreciated that the expression of L1 and L2 from independent plasmids allows the relative levels of L1/L2 VLP production to be optimized for different applications and to obtain molecular structures with optimal delivery properties for different payloads. In some embodiments, a variety of VLP structures can be produced to fit the needs of the different classes of payloads (e.g., DNA, RNA, small molecule, large molecule) both in terms of charge and other functions (e.g. DNA binding domains, VLP inner volume, and endosomal release function): VLPs with a higher content of L2 protein will be better to bind nucleic acids (L2 contains a DNA binding domain) whereas VLPs with a smaller content of L2 protein will be better for other small molecules. VLPs with different ratios of L1:L2 protein will have different inner volumes that will allow a higher concentration of drug to be encapsulated. In some embodiments, the release of payload into the cell will also be modulated. In some embodiments, structures containing more L2 protein may have a higher ability to transfer nucleic acids intracellularly. It should be appreciated that different ratios of L1/L2 may be used. In some embodiments, ratios may he 1:1, 1:2, 1:4, 1:5, 1:20 or 1:100. However, other ratios may be used as aspects of the invention are not limited in this respect.

In some embodiments, each separate expression nucleic acid encodes an L1 (but not an L2) or an L2 (but not an L1) sequence operably linked to a promoter. In some embodiments, other suitable regulatory sequences also may be present. The separate expression nucleic acids may use the same or different promoters and/or other regulatory sequences and/or replication origins, and/or selectable markers. In some embodiments, the separate nucleic acids may be vectors (e.g., plasmids, or other independently replicating nucleic acids). In some embodiments, separate nucleic acids may be independently integrated into the genome of a host cell (e.g., a first nucleic acid integrated and a second nucleic acid on a vector, two different nucleic acids integrated at different positions, etc.). In some embodiments, the relative expression levels of L1 and L2 may be different in different cells, different using different expression sequences, independently regulated, or a combination thereof.

Variant HPV Proteins Having Reduced Immunogenicity:

In some embodiments, an expression vector is used to produce a mutant L1 or L2 protein. In some embodiments, a mutant HPV16L1 protein (called L1*) is expressed along with L2 in a host system (e.g., a 293 cell system). These can then be isolated and assembled as described herein to encapsulate a therapeutic or diagnostic payload (e.g. therapeutic plasmid, siRNA, small molecule drugs, etc.).

In some embodiments, loaded VLPs are produced using certain L1 and/or L2 variant sequences that are not recognized by existing antibodies against HPV (e.g., HPV 16L1) that might be present in patients who have an ongoing HPV infection or who have received the vaccine. It also should be appreciated that loaded VLPs can be produced using L1 and/or L2 proteins that are modified to reduce antigenicity against other HPV serotype antibodies and/or to target the loaded VLP to particular organs or tissues (e.g., lung) or cells or subcellular locations.

Accordingly, certain aspects of the invention relate to methods for loading VLPs with therapeutic, diagnostic or other agents. In certain embodiments, the papilloma virus particles are HPV-VLP. In certain embodiments, the methods described herein utilize HPV-VLPs that contain one or more naturally occurring HPV capsid proteins (e.g., L1 and/or L2 capsid proteins). HPV-VLPs may be comprised of capsid protein oligomers or monomers.

A “VLP” refers to the capsid-like structures which result upon assembly of a HPV L1 capsid protein alone or in combination with a HPV L2 capsid protein. VLPs are morphologically and antigenically similar to authentic virions. VLPs lack viral genetic material (e.g., viral nuclei acid), rendering the VLP non-infectious. VLPs may be produced in vivo, in suitable host cells, e.g., mammalian, yeast, bacterial and insect host cells.

A “capsomere” refers to an oligomeric configuration of L1 capsid protein. Capsomeres may comprise at least one L1 (e.g., a pentamer of L1).

A “capsid protein” refers to L1 or L2 proteins that are involved in building the viral capsid structure. Capsid proteins can form oligomeric structures i.e. pentamers, trimers or be in single units as monomers.

In some embodiments, a VLP can be loaded with one or more medical, diagnostic and/or therapeutic agents, or a combination of two or more thereof. In some embodiments, the methods described herein utilize HPV-VLP that contain one or more variant capsid proteins (e.g., variant L1 and/or L2 capsid proteins) that have reduced or modified immunogenicity in a subject. Examples of variant capsid proteins are described in WO 2010/120266. The modification may be an amino acid sequence change that reduces or avoids neutralization by the immune system of the subject. In some embodiments, a modified HPV-VLP contains a recombinant HPV protein (e.g., a recombinant L1 and/or L2 protein) that includes one or more amino acid changes that alter the immunogenicity of the protein in a subject (e.g., in a human subject). In some embodiments, a modified HPV-VLP has an altered immunogenicity but retains the ability to package and deliver molecules to a subject.

In certain embodiments, amino acids of the viral wild-type capsid proteins, such as L1 and/or L1+L2, assembling into the HPV-VLP, are mutated and/or substituted and/or deleted. In certain embodiments, these amino acids are modified to enhance the positive charge of the VLP interior. In certain embodiments, modifications are introduced to allow a stronger electrostatic interaction of nucleic acid molecules with one or more of the amino acids facing the interior of the VLP and/or to avoid leakage of nucleic acid molecules out of the VLP. Examples of modifications are described in WO 2010/120266. It should be appreciated that any modified HPV-VLP or similar viral vectors (ie. herpes virus vector) may be loaded with one or more agents. Such particles may be delivered to a subject without inducing an immune response that would be induced by a naturally-occurring HPV.

In some embodiments, HPV-VLPs comprise viral L1 capsid proteins. In some embodiments, HPV-VLPs comprise viral L1 capsid proteins and viral L2 capsid proteins. The L1 and/or L2 proteins may, in some embodiments, be wild-type viral proteins. In some embodiments, L1 and/or L2 capsid proteins may be altered by mutation and/or deletion and/or insertion so that the resulting L1 and/or L2 proteins comprise only ‘minimal’ domains essential for assembly of a VLP. In some embodiments, L1 and/or L2 proteins may also be fused to other proteins and/or peptides that provide additional functionality. Examples of modifications are described for example in U.S. Pat. No. 6,991,795, incorporated herein by reference. These other proteins may be viral or non-viral and could, in some embodiments, be for example host-specific or cell type specific. It should be appreciated that VLPs may he based on particles containing one or more recombinant proteins or fragments thereof (e.g., one or more HPV membrane and/or surface proteins or fragments thereof). In some embodiments, VLPs may be based on naturally-occurring particles that are processed to incorporate one or more agents as described herein, as aspects of the invention are not limited in this respect. In certain embodiments, particles comprising one or more targeting peptides may be used. Other combinations of HPV proteins (e.g., capsid proteins) or peptides may be used as aspects of the invention are not limited in this respect.

In some embodiments, viral wild-type capsid proteins are altered by mutations, insertions and deletions. All conformation-dependent type-specific epitopes identified to date are found on the HPV-VLP surface within hyper-variable loops where the amino acid sequence is highly divergent between HPV types, which are designated BC, DE, EF, FG and HI loops. Most neutralizing antibodies are generated against epitopes in these variable loops and are type-specific, with limited cross-reactivity, cross-neutralization and cross-protection. Different HPV serotypes induce antibodies directed to different type-specific epitopes and/or to different loops. Examples of variant capsid proteins are described in WO 2010/120266.

In certain embodiments, viral capsid proteins, HPV L1 and/or L2, are mutated at one or more amino acid positions located in one or more hyper-variable and/or surface-exposed loops. The mutations are made at amino acid positions within the loops that are not conserved between HPV serotypes. These positions can be completely non-conserved, that is that any amino acid can be at this position, or the position can be conserved in that only conservative amino acid changes can be made.

In certain embodiments, L1 protein and L1+L2 protein may be produced recombinantly. In certain embodiments, recombinantly produced L1 protein and L1+L2 protein may self-assemble to form virus-like particles (VLP). Recombinant production may occur in a bacterial, insect, yeast or mammalian host system. L1 protein may be expressed or L1+L2 protein may be co-expressed in the host system.

Cellular hosts that are useful for expressing and purifying HPV L1 and/or L2 recombinant viral capsid proteins are known in the art. For example. HPV L1 and/or L2 proteins may be expressed in Spodoptera frugiperla (Sf21) cells. Baculoviruses encoding the L1 and/or L2 gene of any HPV or recombinant versions thereof from different serotypes (e.g., HPV16, HPV18, HPV31, and HPV58) may be generated as described in Touze et al., FEMS Microbiol. Lett. 2000; 189:121-7; Touze et al., J. Clin. Microbiol. 1998; 36:2046-51); and Combita et al., FEMS Microbiol. Lett 2001; 204(1):183-8. HPV L1 and/or L2 genes may be cloned into a plasmid, such as pFastBac I (Invitrogen). Sf21 cells may be maintained in Grace's insect medium (Invitrogen) supplemented with 10% fetal calf serum (FCS, Invitrogen) and infected with recombinant baculoviruses and incubated at 27° C. Three days post infection, cells can be harvested and VLP can be purified. For example, cells may be resuspended in PBS containing Nonidet P40 (0.5%), pepstatin A, and leupeptin (1 μg/ml each, Sigma Aldrich), and allowed to stand for 30 min at 4° C. Nuclear lysates may then be centrifuged and pellets can be resuspended in ice cold PBS containing pepstatin A and leupeptin and then sonicated. Samples may then be loaded on a CsCl gradient and centrifuged to equilibrium (e.g., 22 h, 27,000 rpm in a SW28 rotor, 4° C.). CsCl gradient fractions may be investigated for density by refractometry and for the presence of L1/L2 protein by electrophoresis in 10% sodium dodecyl sulfate-polyacrylamide gel (SDS-PAGE) and Coomassie blue staining. Positive fractions can be pooled, diluted in PBS and pelleted e.g., in a Beckman SW 28 rotor (3 h, 28,000 rpm, 4° C.). After centrifugation, VLP can be resuspended in 0.15 mol/L NaCl and sonicated, e.g., by one 5 second burst at 60% maximum power. Total protein content may be determined.

Viral capsid proteins may also be expressed using galactose-inducible Saccharomyces cerevisiae expression system. Leucine-free selective culture medium used for the propagation of yeast cultures, yeast can be induced with medium containing glucose and galactose. Cells can be harvested using filtration. After resuspension, cells may be treated with Benzonase and subsequently mechanically disrupted (e.g., using a homogenizer). Cell lysate may be clarified using filtration. An exemplary protocol can be found in Cook et al. Protein Expression and Purification 17, 477-484 (1999).

Buck et al. (J. Virol. 78, 751-757, 2004) reported the production of papilloma virus-like particles (VLP) and cell differentiation-independent encapsidation of genes into bovine papillomavirus (BPV) L1 and L2 capsid proteins expressed in transiently transfected mammalian cells, 29311 human embryonic kidney cells, which stably express SV40 large T antigen to enhance replication of SV40 origin-containing plasmids. Pyeon et al. reported a transient transfection method that achieved the successful and efficient packaging of full-length HPV genomes into HPV16 capsids to generate virus particles (PNAS 102, 9311-9316 (2005)). Transiently transfected cells (e.g., 293 cells, for example 293T or 293TT cells) can be lysed by adding Brij58 or similar nonionic polyoxyethylene surfactant detergent, followed by benzonase and exonuclease V and incubating at 37° C. for 24 h to remove unpackaged cellular and viral DNA and to allow capsid maturation. The lysate can be incubated on ice with 5 M NaCl and cleared by centrifugation. VLP can be collected by high-speed centrifugation.

Capsid proteins may also be expressed in E. coli. In E. coli, one important potential contaminant of protein solutions is endotoxin, a lipopolysaccharide (LPS) that is a major component of the outer membrane of Gram-negative bacteria (Schädlich et al. Vaccine 27, 1511-1522 (2009)). For example, transformed BL21 bacteria may be grown in LB medium containing 1 mM ampicillin and incubated with shaking at 200 rpm at 37 C. At an optical density (OD₆₀₀ nm) of 0.3-0.5, bacteria can be cooled down and IPTG may be added to induce protein expression. After 1.6-18 h bacteria may be harvested by centrifugation. Bacteria may be lysed by homogenizing, lysates may be cleared, capsid proteins purified and LPS contamination removed, using e.g., chromatographic methods, such as affinity chromatography and size exclusion chromatography. L1′S contamination may also be removed using e.g., 1% Triton X-114.

In certain embodiments, VLPs are loaded with the one or more therapeutic agents. After isolation of L1 and L2 capsid proteins which may be in the form of monomers or oligomers, VLPs may be assembled and loaded by disassembling and reassembling L1 or L1 and L2 viral capsid proteins, as described herein. Salts that are useful in aiding disassembly/reassembly of viral capsid proteins into VLPs, include Zn, Cu and Ni, Ru and Fe salts. In some embodiments, VLPs may be loaded with one or more therapeutic agents.

Loading of VLPs with agents utilizing a disassembly-reassembly method has been described previously, for example in U.S. Pat. No. 6,416,945 and WO 2010/120266, incorporated herein by reference. Generally, these methods involve incubation of the VLP in a buffer comprising EGTA and DTT. Under these conditions, VLP completely disaggregated into structures resembling capsid proteins in monomeric or oligomeric form. A therapeutic or diagnostic agent, as described herein, may then be added and the preparation diluted in a buffer containing DMSO and CaCl₂ with or without ZnCl₂ in order to reassemble the VLP. The presence of ZnCl₂ increases the reassembly of capsid proteins into VLP. In some embodiments, one or more of these reassembly methods may be used to assemble capsid proteins to form VLPs that encapsulate one or more agents without requiring an initial VLP disassembly procedure, as described herein.

In certain embodiments, VLP are loaded with the one or more therapeutic agents. After isolation of L1 and L2 capsid proteins, these may mixed directly after purification from the host cell with the therapeutic agent and reassembled into loaded VLPs as described herein, the preparation diluted in a buffer containing DMSO and CaCl₂ with or without ZnCl, in order to reassemble the VLP. The presence of ZnCl₂ increases the reassembly of capsid proteins into VLP.

It was surprisingly found that certain ratios of a) Capsid protein to reaction volume, b) agent to capsid protein, and/or c) agent to reaction volume lead to agent-loaded VLP (VLP comprising entrapped agent) that exhibit superior delivery of agent to target cells when compared to agent-loaded VLP prepared using previously described methods. VLP loaded with agents using the methods described herein, in certain embodiments, are able to deliver agent to 65%, 75%, 85%, 95%, 96%, 97%, 98%, or 99% of target cells. One non-limiting example of the improved method is exemplified in the Examples.

For example, VLP may be loaded with a nucleic acid using a method comprising: a) contacting a preparation of capsid proteins with the nucleic acid in a reaction volume, wherein i) the ratio of capsid protein to reaction volume ranges from 0.1 μg capsid protein per 1 μl reaction volume to 1 μg capsid protein per 1 μl reaction volume; ii) the ratio of nucleic acid to capsid protein ranges from 0.1 μg nucleic acid per 1 μg capsid protein to 10 μl nucleic acid per 1 μg capsid protein; and/or iii) the ratio of nucleic acid to reaction volume ranges from 0.01 μg nucleic acid per 1 μl reaction volume to 10 μg nucleic acid per 1 μl reaction volume, and b) reassembling the capsid proteins to form a VLP, thereby encapsulating the nucleic acid within the VLP. In other embodiments, the ratio of HPV-capsid protein to reaction volume ranges from 0.2 μg HPV-capsid protein per 1 μl reaction volume to 0.6 μg HPV-capsid protein per 1 μl reaction volume. In yet other embodiments, the ratio of nucleic acid to HPV-capsid protein ranges from 0.5 μg nucleic acid per 1 μg HPV-capsid protein to 3.5 μg nucleic acid per 1 μg HPV-capsid protein. In yet other embodiments, the ratio of nucleic acid to reaction volume ranges from 0.2 μg nucleic acid per 1 μl reaction volume to 3 μg nucleic acid per 1 μl reaction volume.

The step of dissociating the VLP or capsid protein oligomers can be carried out in a solution comprising ethylene glycol tetraacetic acid (EGTA) and dithiothreitol (DTT), wherein the concentration of EGTA ranges from 0.3 mM to 30 mM and the concentration of DTT ranges from 2 mM to 200 mM. In certain embodiments, the concentration of EGTA ranges from 1 mM to 5 mM. In certain embodiments, the concentration of DTT ranges from 5 mM to 50 mM.

The step of reassembling of capsid proteins into a VLP can be carried out in a solution comprising dimethyl sulfoxide (DMSO), CaCl₂ and ZnCl₂, wherein the concentration of DMSO ranges from 0.03% to 3% volume/volume, the concentration of CaCl₂ ranges from 0.2 mM to 20 mM, and the concentration of ZnCl₂ ranges from 0.5 μM to 50 μM. In certain embodiments, the concentration of DMSO ranges from 0.1% to 1% volume/volume. In certain embodiments, the concentration of ZnCl₂ ranges from 1 μM to 20 μM. In certain embodiments, the concentration of CaCl₂ ranges from 1 mM to 10 mM.

In certain embodiments, the loading method is further modified to stabilize the VLP, in that the loading reaction is dialyzed against hypertonic NaCl solution (e.g., using a NaCl concentration of about 500 mM) instead of phosphate-buffered saline (PBS), as was previously described. Surprisingly, this reduces the tendency of the loaded VLP to form larger agglomerates and precipitate. In certain embodiments, the concentration of NaCl ranges between 5 mM and 5 M. In certain embodiments, the concentration of NaCl ranges between 20 mM and 1 M.

Aspects of the invention are not limited in its application to the details of construction and the arrangement of components set forth in the preceding description or illustrated in the examples or in the drawings. Aspects of the invention are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1

### Production and Purification of Capsid Proteins in Host Cells and In Vitro Reassembly into VLPs

Suspension cultures of Sf9 insect cells were maintained in serum-free Sf-900™ II medium (Invitrogen, Lide Technologies) and expanded from shake flasks to WAVE bioreactors™ (GE Healthcare Lifesciences). Approximately 2 L of shake flask culture was utilized to seed the 10 L WAVE bioreactors™ at an initial density of 4×10⁵ cells/ml.

Once the actively growing culture reached a density between 1.5-2×10⁶ cells nil, it was infected with a recombinant baculovirus stock for HPV 16L1 or HPV 16/31 mutant and a HPV16L2 at an MOI of 5. Recombinant baculovirus stocks were produced, as described herein (Table 1).

According the present invention, an overview of an exemplary protocol for generating Baculovirus generation and preparing a high-titer stock preparation is described as follows. Transform DH10Bac Competent Cells with pFastBac construct and heat shock the mixture. Serial dilute the cells using SOC medium to 1:10, 1:100 and 1:1000 dilutions. Grow cultures for 4 hours at 37 C. at 250 rpm. Streak the 1:10, 1:100 and 1:1000 dilutions onto selective plates of LB-Agar/Kan/Tet/Gent/X-gal/PTG. Incubate plates for 48 hours at 37 C. Select three white colonies. Grow each culture O/N at 37 C. at 250 rpm in LB plus Kan, Gent. & Tet. Harvest cell pellets by centrifugation and isolate recombinant Bacmid by alkaline lysis method. Determine Bacmid concentration by 260:280. Tranfect Sf9 cells with Bacmid/cellfectin complex and plate. Incubate plates for four days in a humidified 27 C. tissue culture incubator. Transfer conditioned media to 30 ml SF Sf9 culture. Grow culture 3-5 days. Monitor for cell viability and cell diameter using Vi-Cell. Harvest conditioned media and cell pellet when viability is less than 75%. Perform titer (BacPAK RapidTiter Kit) and Western Plot analysis. Expand recombinant virus by infecting a 1 L culture of Sf9 cells at an MOI of 0.1 with the best expressing Baculovirus clone. Harvest conditioned media by centrifugation once viability has dropped less than 75%. Perform titer analysis using RapidTiter Kit.

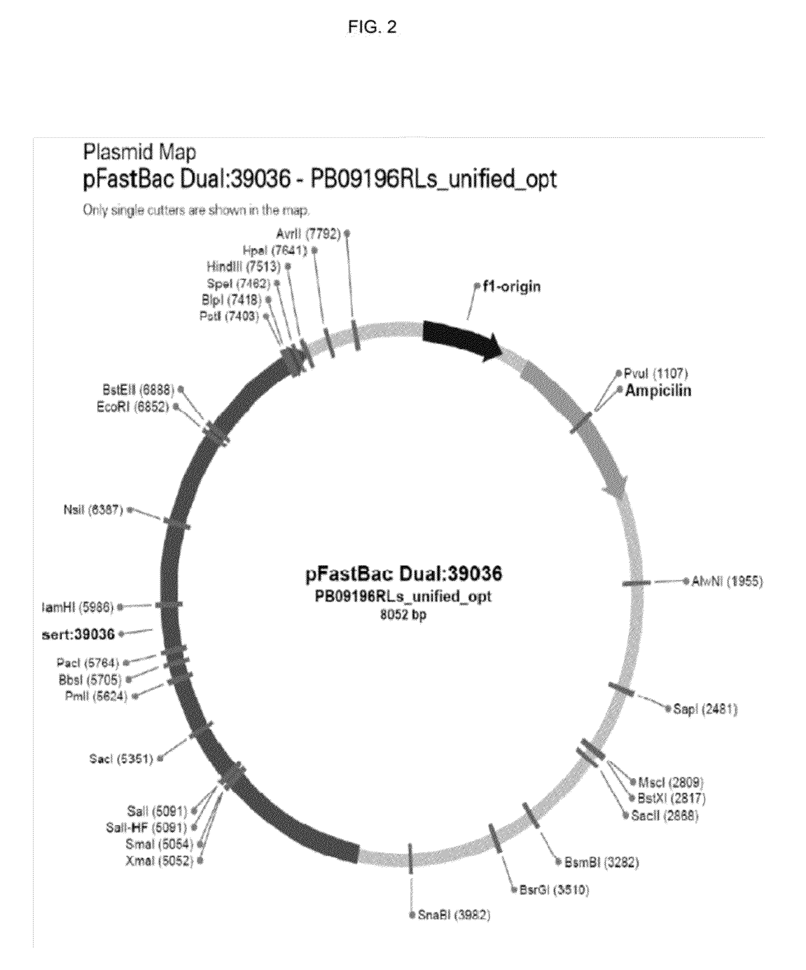

To generate the recombinant baculovirus for HPV16/31L1 production, the pFastBac™ plasmid (Invitrogen, Life Technologies) (FIG. 2) containing 16/31 L1 DNA sequence (SEQ ID NO: 1) was used. To generate the recombinant baculovirus for HPV16L2 production, the pFastBac™ plasmid containing L2 DNA sequence (SEQ ID NO: 2) was used. During recombinant protein production, the bioreactor was monitored daily for cell count, viability, cell size and pH. Seventy-two hours post-infection, the cell pellet was obtained by tangential-flow filtration, washed in PBS, re-pelleted by centrifugation, and stored at −80° C. Western blot using protein-specific antibodies for L1 and L2 proteins were then used to verify the presence of the recombinant protein.

Following verification of expression, purification of HPV capsomeres produced above was performed. Cells were thawed on ice and then resuspended in ice-cold lysis buffer (PBS plus 0.5% Nonidel™ P-40 (Shell Chemical Co.)) at a ratio of 10 ml of buffer per gram of cell

TABLE 1
Transform DH10Bac Cells with pFastbac Construct
Use pFastbac Dual construct generated at DNA2.0 to transform DH10Bac cells by heat shock
method (i.e. 1 ng, pFactbac construct in 100 ul of cells. Incubate for 30 minutes on ice. Heat
at 42 C. for 45 seconds. Chill on ice for two minutes). Grow cultures at 37 C., 225 rpm in SOC
media for four hours. Prepare 1:10, 1:100, and 1:1000 dilutions of culture. Plate dilutions on
Bac-to-Bac selective plates. Incubate plates at 37 C. for two days.
Purify Recombinant Bacmid
Select three well defined white colonies from the Bac-to-Bac selective plates and culture the
cells in selective LB media overnight. Collect bacterial cells by centrifugation (14K × g. 3
minutes). Resuspend cell pellets in P1 buffer. Lyse cells by the addition of an equal volume
of P2 buffer. Incubate at room temperature for five minutes. Precipitate genomic DNA and
protein by addition of a half colume of P3 bugger and incubation on ice for five minutes.
Remove precipitated contaminants by centrifugation (14K × g; 10 minutes) and reserve
supernatant. Precipitate the bacmid by addition of an equal volume of Isopropanol followed
by an overnight incubation at 20 C. Pellet bacmid by centrifugation. Wash pelleted bacmid
with 70% ethanol. Let pellet air dry. Resuspend pellet in TE. Determine yield and purity by
OD260-OD280.
Transfect Sf9 Cells With Recombinant Bacmid
For each bacmid prepare a 6-well plate with 1 × 20e6 cells per well in standard growth media
(i.e. Sf-900 II). Allow cells to attach to the plate for at least 1 hour. In a BSC, prepare
bacmid Cellfectin complex by mixing 1 ug of bacmid that has been diluted with 100 ul of
Grace's media with 6ul of cellfectin transfection reagent that has been diluted with 100 ul of
Grace's media. Let complexes form for 30 minutes at room temperature. Remove media
from the cells in upper left corner well, dilute bacmid cellfectin complex with 800 ul of
Grace's media, add transfection solution to the upper left corner well. Place plates into a
humidified incubator at 27 C. After five hours, remove transfection solution from the cells in
the upper left corner well and add 2 ml of growth media (i.e. Sf-900 II). Return plates to the
humidified incubator. Check cells daily under a microscope to confirm transfection (cells
should not grow as fast as control cells and should increase in diameter, and eventually the
cells should show signs of lysing). After four days, harvest P0 viral stock (i.e. conditioned
media from upper left corner well).
Amplify P0 Baculoviral Stock:
For each baculoviral stock, add 1 ml of the P0 viral stock to a 30 ml culture in a 125 ml shake
flask of Sf9 cell at a cell density of 1e6 cells/ml. An additional SF is utilized as a negative
control and 1 ml of growth media added. Shaking incubator parameters are 120 rpm and
27.5 C. Cultures are monitored daily with the Vi-Cell for cell density, cell viability, and
diameter. In a proper infection, within 48 hours the insect cell culture should have
significantly lower cell density and cell viability and increased cell diameter. Cultures are
maintained for three to five days and harvested by centrifugation (2500 × g, 10 minutes) once
viability has dropped below 75%. Transfer the conditioned media (P1) viral stock to a fresh
tube and store at 4 C. Reserve cell pellet for Western analysis. Determine titer for the p1
viral stock using the Clontech BacPAK Rapid Titer Ket according to manufacturer's
protocol.
Expand P1 Baculoviral Stock
For the best expressing baculoviral stock (i.e. Western Analysis), add 1.5e8 pfu of P1 viral
stock to a 1 L culture of Sf9 cells in a 3 L Shake Flask at 1.5e6 cells per ml (i.e. MOI of 0.1).
Shaking incubator parameters are 120 rpm and 27.5 C. Cultures are monitored daily with Vi-
Cell for cell density, cell viability, and cell diameter. Cultures are maintained for two to five
days and harvested by centrifugation (2500 × g, 10 minutes) once viability has dropped below
75%. Transfer the conditioned media (P1) viral stock to a fresh sterile bottle and store at 4 C.
Determine titer for the P2 viral stock using the Clontech BacPAK Rapid Titer Kit according
to manufacturer's protocol.

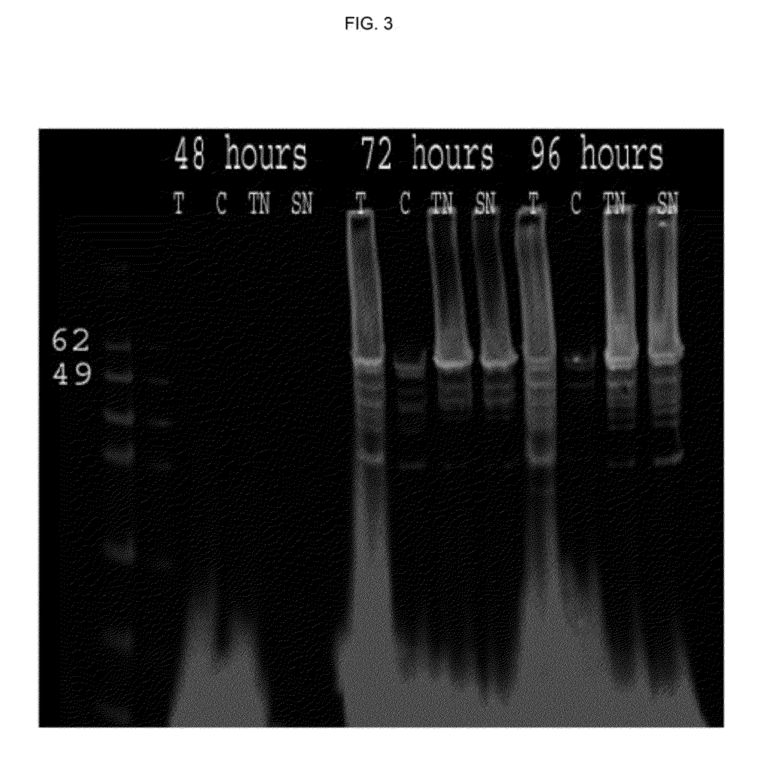

paste. Resuspended cells were then incubated on ice for 15 min. After chemical lysis, nuclei were isolated by centrifugation (3000×g for 15 min) and then resuspended in ice-cold PBS without detergent. Capsid proteins were then solubilized from the isolated nuclei with three 15 s bursts of a sonicator at 50% maximal power. Insoluble material was then clarified by centrifugation (1000×g for 10 min) and the resulting supernatant was diatiltered into TMAE buffer by TFF using a 100 kDa molecular weight cut-off filter. Western Blot was used to demonstrate that the majority of the capsid proteins were localized in the nuclear fraction. (FIG. 3)

Capsid proteins were then loaded onto a TMAE column, washed, and eluted using a linear salt gradient. Early fractions containing the proteins of interest were then pooled, dialyzed into disassociation buffer, and concentrated to a final concentration of 1 mg/ml.

Purified capsid proteins were then assembled in a cell free system together with a plasmid (pENTRT™/U6 plasmid (Invitrogen, Life Technologies)) expressing an shRNA construct containing the short hairpin RNA sequence generated using primer sequences (SEQ ID NO: 3 and SEQ ID NO: 4) to create VLP encapsulating theshRNA using the following loading protocol.

Loading Protocol

In a clean 15 ml conical tube the following reagents were added and incubated at 37° C. for 30 min: 200 μg of capsomere protein; 100 μg pENTRT™/U61shRNA plasmid; 0.5 μl DMSO; and 15 μl Solution 2 (150 mM Tris-HCl pH7.5, 450 mM NaCl, 330 μl dH₂O), brought up to a total volume of 150 μl.

Solution 3 (2 mM CaCl₂, 5 μM CaCl₂, 50 mM Tris-HCl pH 7.5, 150 mM. NaCl, 434 μl dH₂O) was then added to the above mixture and incubated at 37° C. for 30 min.

Solution 4 (4 mM CaCl₂, 10 μM CaCl₂, 50 mM Tris-HCl pH 7.5, 150 mM NaCl, 1224 μl dH₂O) was then added to the above mixture and incubated at 37° C. for 2 hrs.

The mixture was then dialyzed in 1×PBS at 4° C. overnight.

### Example 2

### Production of Mutant L1* and L2 Capsid Proteins in Mammalian Cell System

Similarly to Example 1 described above, a mammalian culture system is used to produce mutant L1*(16/31) and L2 capsid proteins. Plasmids containing human-optimized codon sequences are used for this purpose (SEQ ID NO: 5) and a general protocol is followed (Buck, C. B., et al. (2005) Methods Mol. Med., 119: 445-462, which reference is incorporated herein).

### Example 3

### Assembly into VLPs from Capsid Proteins

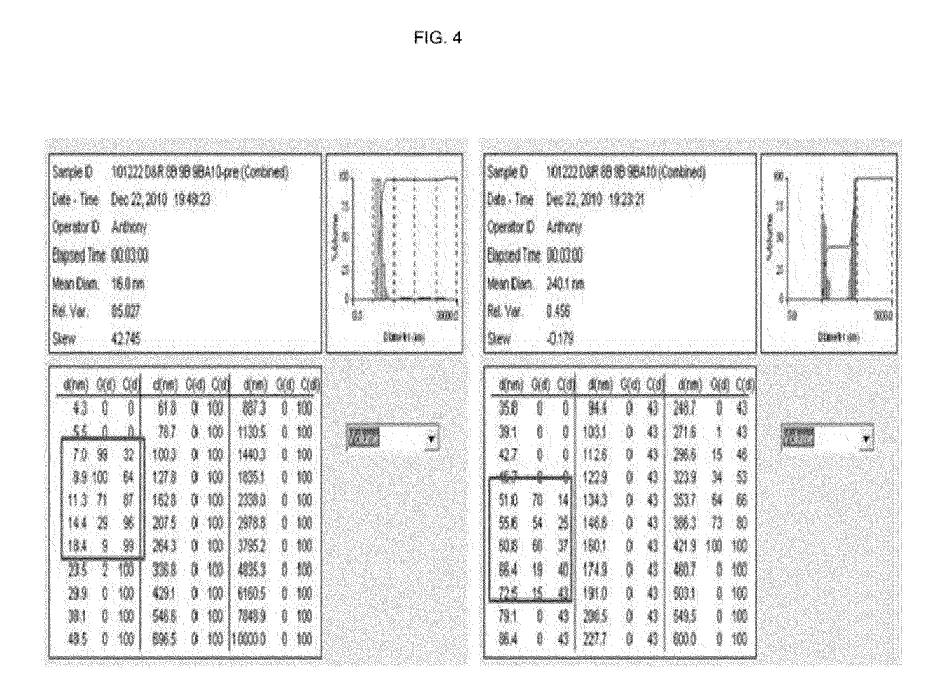

Capsid proteins isolated from insect cells were assembled into VLPs as described. Dynamic light scattering (DLS) demonstrates presence of capsid proteins in monomeric and oligomeric forms (<10 nm) after harvest and prior to the loading procedure. After the reassembly in presence of the nucleic acid payload, VLPs are seen by DLS (50-70 nm diameter) (FIG. 4).

### Example 4

### Functional Transfer of Luciferase Expression

Results show functional transfer of luciferase expression. VLPs were generated using different production methods to compare efficacy. Transfection of luciferase plasmid (pClucF) using standard lipofectamine transfection at various plasmid amounts (0.1 ng/well, ng/well, 10 ng/well) was used to create a range of positive controls. 10 ng of pClucF plasmid was used without transfection reagent as a reagent/background control.

AB1-2 refers to HPV16L1L2 VLP generated using the methods described above, where a single plasmid like p16sheLL (SEQ ID NO: 6) was used to co-express wildtype HPV L1 and 1.2 proteins.

Capsid proteins were purified, as described above, from 293 cells transfected with the co-expression plasmid for L1 and L2. Capsid proteins were then subjected to the following loading protocol, thereby forming loaded VLP.

Loading Protocol

In a clean 15 ml conical tube the following reagents were added and incubated at 37° C. for 30 min: 200 μg of capsid proteins, 100 μg pClucF, 0.5 μl DMSO, 15 μl Solution 2

(150 mM Tris-HCl 017.5, 450 mM NaCl, 330 μl dH₂O), brought up to a total volume of 150

Solution 3 (2 mM CaCl₂, 5 μM CaCl₂, 50 mM Tris-HCl pH 7.5, 150 mM NaCl, 434 μL dH₂O) was then added to the above mixture and incubated at 37° C. for 30 min.

Solution 4 (4 mM CaCl₂, 10 μM CaCl₂, 50 mM Tris-HCl pH 7.5, 150 mM NaCl, 1224 μl dH₂O) was then added to the above mixture and incubated at 37° C. for 2 hrs.

The mixture was then dialyzed in 1×PBS at 4° C. overnight.

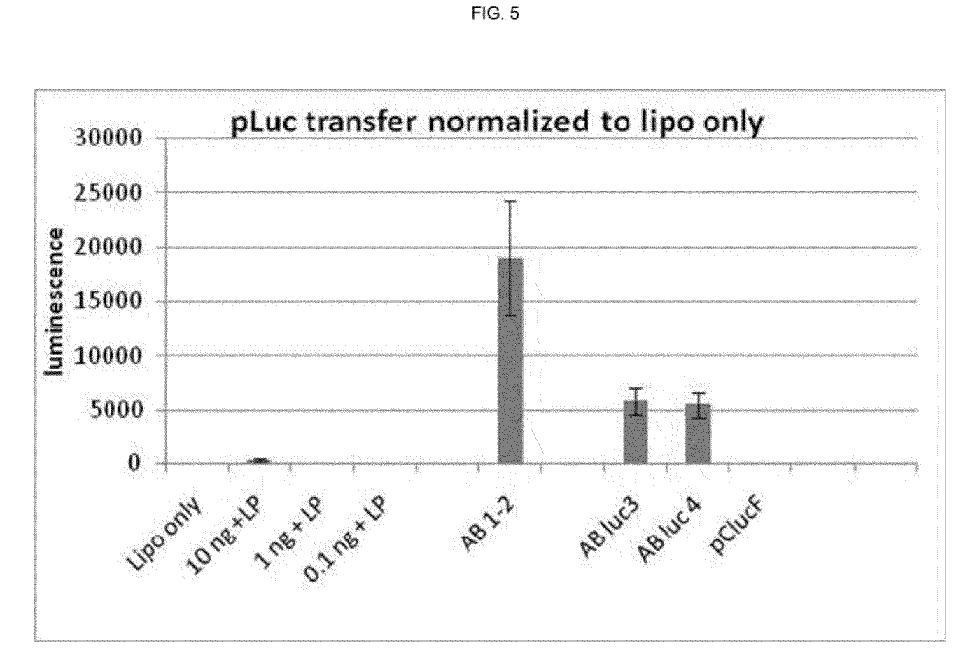

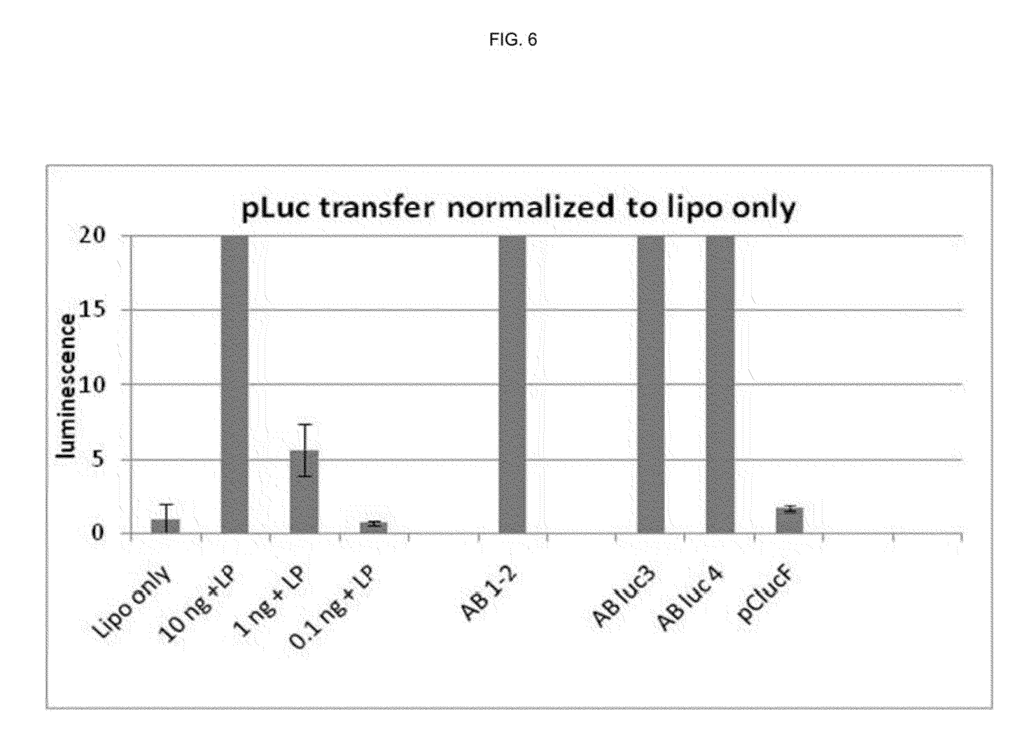

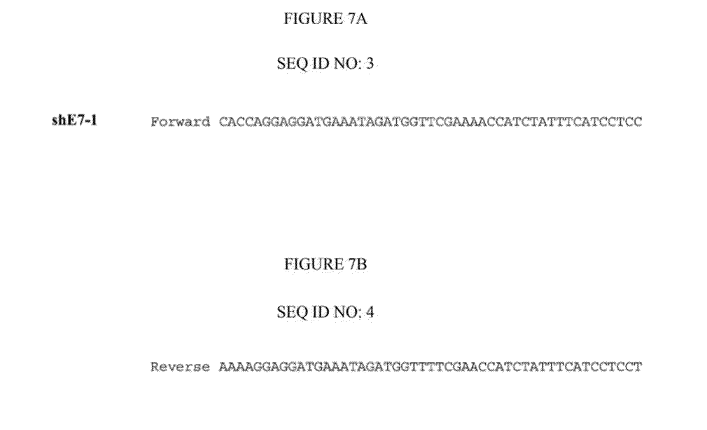

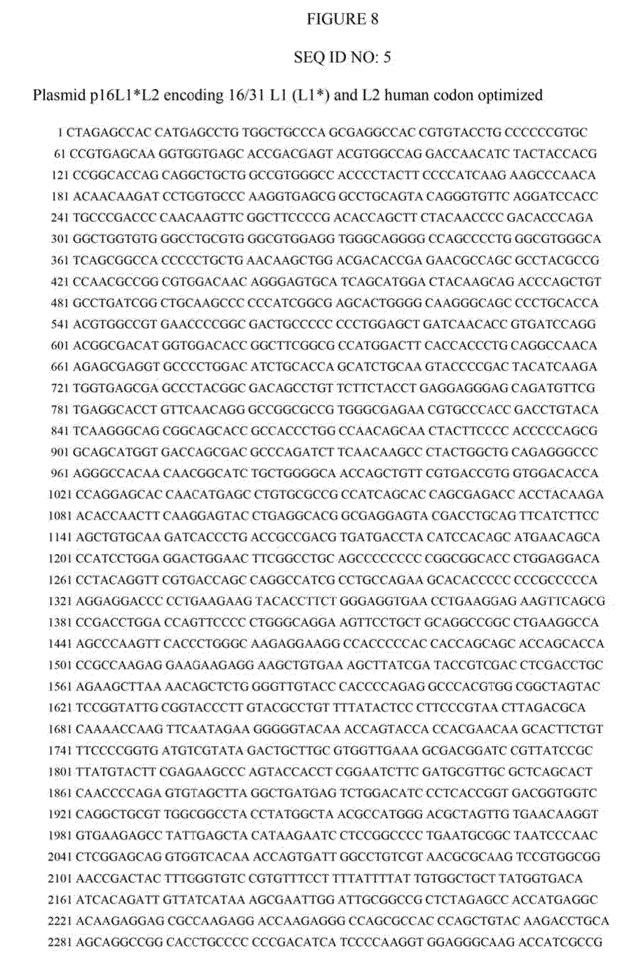

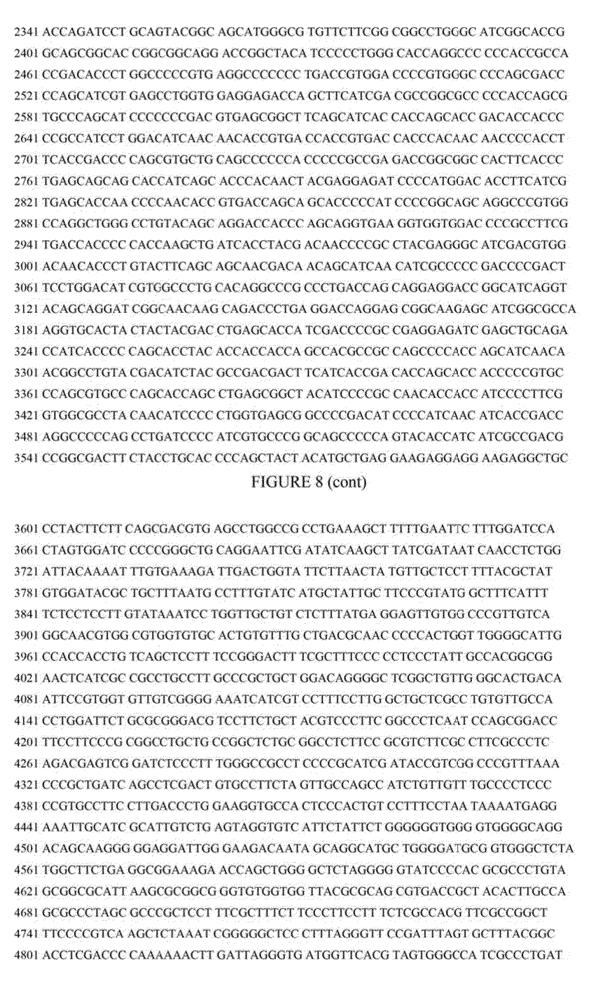

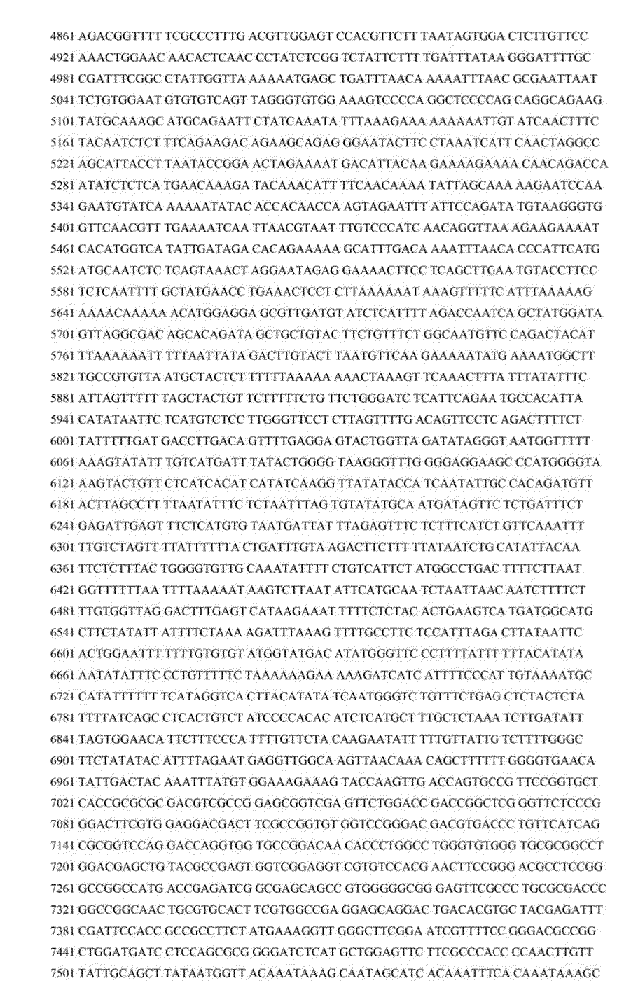

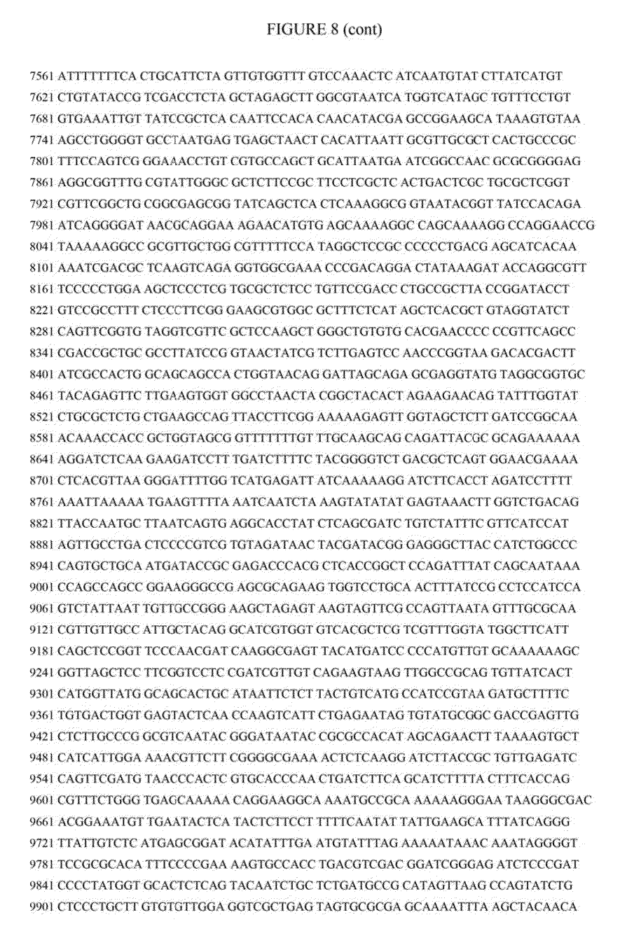

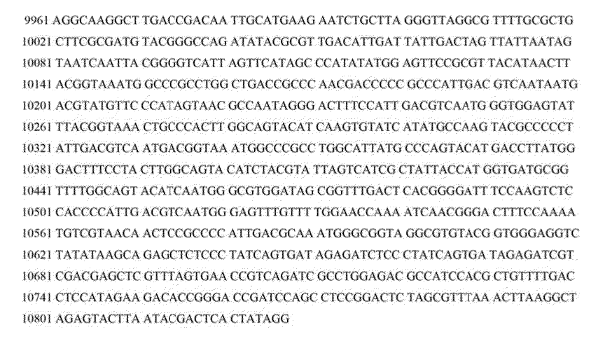

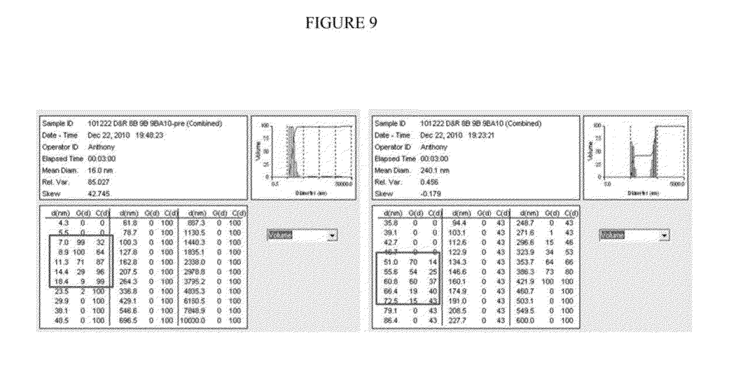

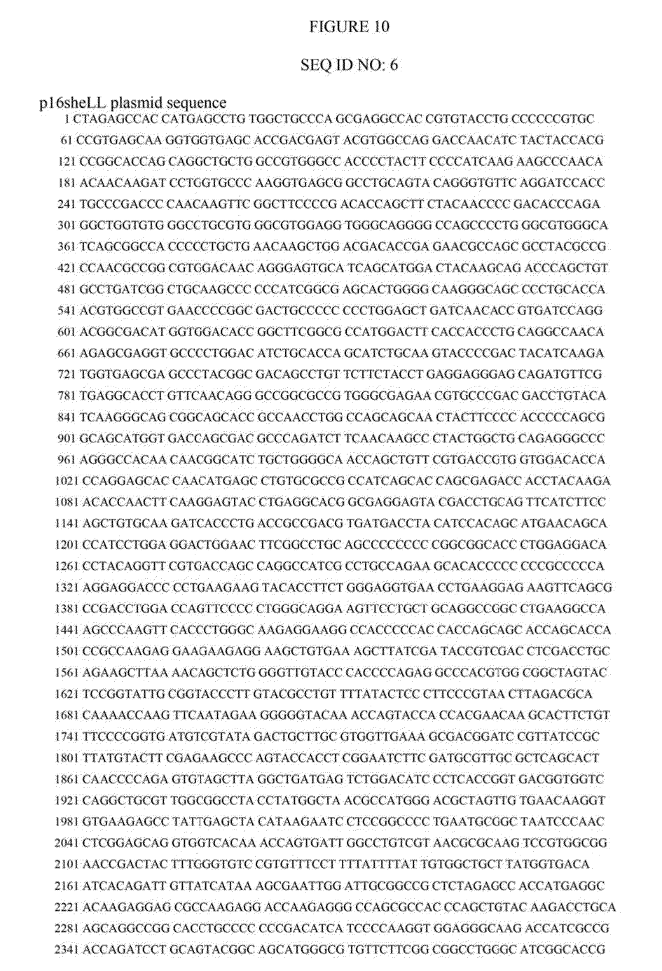

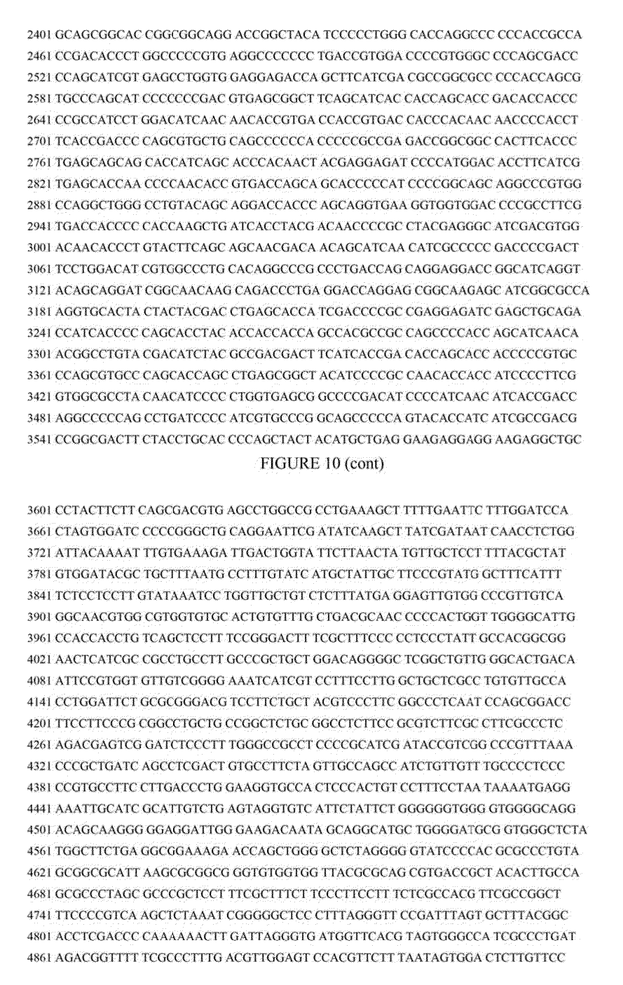

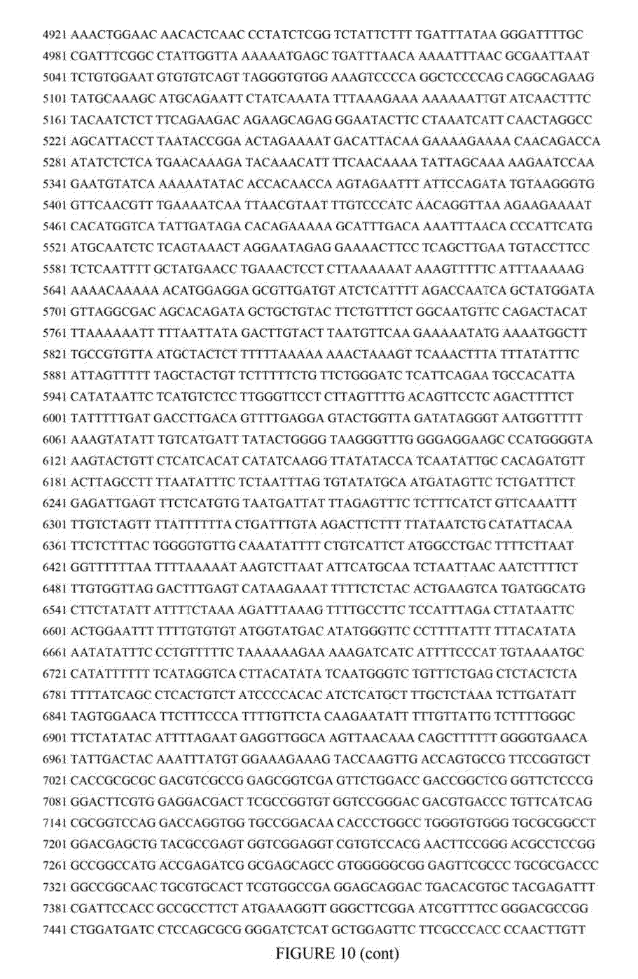

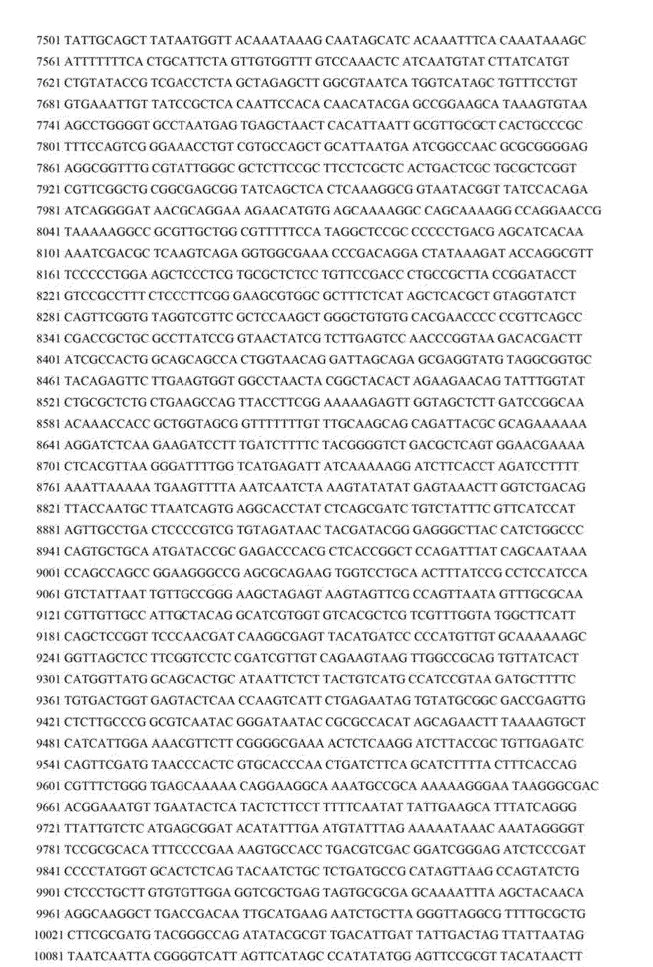

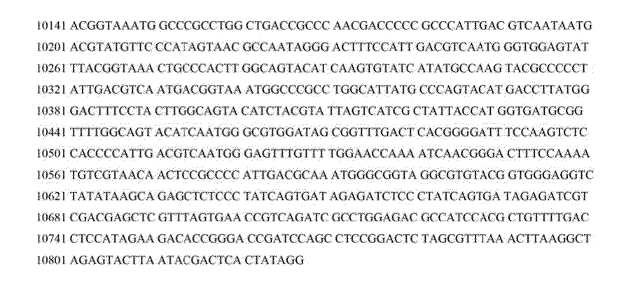

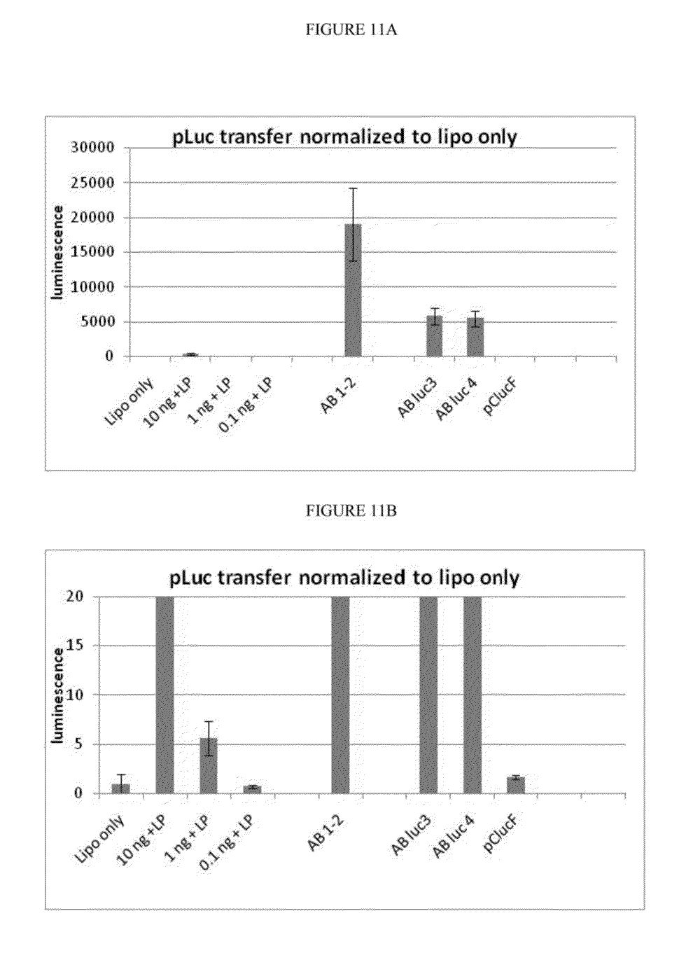

Loaded VLP were then used to treat Hela cells in 96 well plates and luciferase signal was read after 48 hrs (Table 2, FIGS. 5 and 6).

AB luc3 and AB luc4 were produced in 293 cells after transfection with the pl6sheLL plasmid as pseudovirions (PSV) already encapsulating the payload plasmid (pClucF) (Buck, C. B., et al. (2005) Methods Mol. Med., 119: 445-462). Results showed superior transfer of plasmid when the reassembly loading method was used (AB 1-2) compared with VLPs that were loaded through packaging of plasmid in the host cells (AB luc 3 and AB luc 4).

TABLE 2

| Sample | Average | STDEV |
|---|---|---|
| Lipo only | 1 | 1 |
| 10 ng + LP | 338.4552177 | 114.5688758 |
| 1 ng + LP | 5.61254622 | 1.747839908 |
| 0.1 ng + LP | 0.732641742 | 0.135130943 |
| AB 1-2 | 19011.91454 | 5216.078827 |
| AB luc3 | 5769.104355 | 1178.278814 |
| AB luc 4 | 5487.777321 | 1115.096887 |
| pClucF | 1.639379622 | 0.218550273 |

TABLE 3
Materials

| Item | Manufacturer | Catalog |
|---|---|---|
| pFastbac Dual: 39036 | DNA 2.0 | 39036 |
| (PB09196RLs_unified_opt) | | |
| Bac-to-Bac Dual vector | Invitrogen | 10712024 |
| MAX Efficiency Chemically | Invitrogen | 10361-012 |
| Competent DH10Bac | | |
| LB Broth | Amresco | J106 |
| Agar | Amresco | J637 |
| Kanamycin Sulfate | Calbiochem | 420311 |
| Gentamicin | Gibco | 15710 |
| Tetracycline Hydrochloride | Sigma | T7660 |
| Bluo-gal | Invitrogen | 15519-028 |
| Isopropylthis-B-galactoside | Inalco | 1758-1400 |
| (IPTG) | | |
| RNase A | | |
| P1 Buffer | Qiagen | 1014858 |
| P2 Buffer | Qiagen | 1014950 |
| P3 Buffer | Qiagen | 1014965 |
| Isopropanol | Malinkrodt | 3032-22 |
| Ethanol | Signma | E7023 |
| TE Buffer | Qiagen | 1018456 |
| Cellfectin reagent | Invitrogen | 10362-010 |
| Sf9 Cells | Gibco | 11496-015 |
| Sf-900 II SFM | Gibco | 10902-096 |
| Grace's Insect Cell Culture | Gibco | 11595-030 |
| Medium | | |
| BacPak Rapid Titer Kit | Clontech | 631406 |
| Mouse anti-6XHis antibody | Clontech | 631212 |
| Qdot 800 goat anti-mouse IgG | Invitrogen | Q1107MP |
| conjugate | | |
| Acetone | J. T. Baker | 9002-03 |
| Formaldehyde | VWR | VW3408-1 |
| Dimethylformamide | Sigma-Aldrich | 319937 |

TABLE 4
Equipment

| Item | Manufacturer/Model | Equipment # |
|---|---|---|
| Microbial Biosafety Cabinet | Forma Scientific/1184 | PB0138 |
| Shaking Microbial Incubator | NBS/PsycroTherm | PB0045 |
| Microcentrifuge | Eppendorf/5415D | PB0159 |
| UV/Vis Spectrophotometer | Agilent 8453 | PB0090 |
| Insect Biosafety Cabinet | Baker Co./SterilGARD III | 5007-0000 |
| Humidified Incubator | Forma Scientific/3326 | PB0013 |
| Microscope | Olympus/1X70 | PB0075 |
| Shaking Insect Incubator | NBS/Innova 4000 | PB0044 |
| Cell Analyzer | Beckman Coulter/Vi-Cell | PB0085 |
| | XR | |
| Table Top Centrifuge | Beckman/Allegra X-15R | PB0160 |
| Western Imaging Station | Li-Cor/Odyssey | PB0073 |

While the above descriptions regarding the present invention contains much specificity, these should not he construed as limitations on the scope, but rather as examples. Many other variations are possible. Accordingly, the scope should be determined not by the embodiments illustrated, but by the appended claims and their legal equivalents. For example, alternative viral vectors may be used in place of the betapapillomavinis. For example, an alternative viral vectors may include herpes virus vectors.

We claim:

1. A composition for transdermal drug delivery for the treatment of Psoriasis consisting essentially of virus-like protein and RNA which inhibits expression of cytokines.

2. A composition of claim 1, wherein the RNA comprises siRNA.

3. The composition of claim 1, wherein the siRNA inhibits the expression of TNF-α.

4. The composition of claim 3, wherein the virus-like protein is comprised of a HPV protein.

5. The composition of claim 3, wherein the virus-like protein is comprised of a herpes virus protein.

6. The composition of claim 4, wherein the HPV protein is L1 or L2.

7. The composition of claim 4, wherein the HPV protein is L1 and L2.

8. The composition of claim 4, wherein the HPV is from the genus betapapillomavirus.

9. (canceled)

10. The composition of claim 4, where in the HPV protein is HPV5.

11. The composition of claim 4, wherein the HPV protein is HPV5.
